(19) United States
(12) Patent Application Publication
(10) Pub. No.: US 2026/0200823 A1
(43) Pub. Date: Jul. 16, 2026

(54) METHOD FOR PRODUCING REGENERATED TEREPHTHALIC ACID CHARACTERIZED BY NOT USING ORGANIC SOLVENT AND REGENERATED TEREPHTHALIC ACID PRODUCED THEREBY

(71) Applicant: Terracle Co., Ltd., Busan (KR)

(72) Inventors: Seong Eon LEE, Incheon (KR); Do Wan KIM, Incheon (KR)

(21) Appl. No.: 19/380,836

(22) Filed: Nov. 5, 2025

(30) Foreign Application Priority Data
Jan. 14, 2025 (KR) 10-2025-0005639

(51) Int. Cl. C07C 51/41; C07C 51/42; C07C 63/26

(52) U.S. Cl. CPC C07C 51/41; C07C 51/42; C07C 63/26

(57) ABSTRACT

Provided are a method for producing regenerated terephthalic acid characterized by not using an organic solvent including mixing waste polyester and an alkaline catalyst, heating the mixture to 80 to 100° C. and sonicating the mixture at a frequency of 20 to 60 kHz and a power of 100 to 5,000 W under 1 atm conditions for 30 minutes to 5 hours, separating a terephthalic acid metal salt by adding water to the sonicated mixture, and treating the separated terephthalic acid metal salt with an acidic solution to precipitate regenerated terephthalic acid, and regenerated terephthalic acid produced thereby.

### CROSS-REFERENCE TO RELATED APPLICATION

This application claims priority to and the benefit of Korean Patent Application No. 10-2025-0005639, filed on Jan. 14, 2025, the disclosure of which is incorporated herein by reference in its entirety.

### BACKGROUND

### Field

The present disclosure relates to a method for efficiently recovering terephthalic acid from waste polyester using an alkaline catalyst and ultrasonic irradiation without using an organic solvent. The present disclosure relates to a method for increasing the production efficiency of terephthalic acid by separating terephthalic acid from waste polyester with high efficiency and high purity, being economical without requiring a separate impurity removal process by not using an organic solvent such as toluene or xylene, and accelerating reactions and promoting uniform decomposition through ultrasonic irradiation.

### Description of the Related Art

Polyesters are polymers which are the most widely purchased and widely utilized worldwide, and mass-produced in excess of 75 million tons annually. The polyester may be produced at a relatively low cost and is used for various applications, including clothing, carpets, and films due to physical, chemical, and thermal properties. Among the polyesters, polyethylene terephthalate (PET) is one of polyesters which is widely used in various methods, including disposable applications such as beverage containers. Among the polyesters, particularly, as PET has become commercially successful, efforts have been made to recover materials from post-consumption, post-industrial use, scraps, and other sources, and to reuse these materials as an alternative of basic disposal methods such as landfill. As the annual global volume of plastic waste made of these polyesters is difficult to handle, interest in recycling waste polyester or a regeneration process using waste polyester is increasing.

One of methods for chemically recycling waste polyester is a depolymerization technology, which is a technique of converting polyester into monomers and re-polymerizing the monomers generated by depolymerization to reproduce new products. These depolymerization techniques may be divided into glycolysis, methanolysis, and hydrolysis.

Pure polyester has a low impurity content to easily synthesize terephthalic acid through depolymerization, but waste polyester is highly likely to contain various foreign substances including heavy metals to require an additional separation process for removing foreign substances, and has a low conversion rate, which is economically disadvantageous.

In the case of the waste polyester, generally, polyester polymer chains are effectively dissolved using organic solvents to promote the decomposition reaction, and these organic solvents are characterized by selectively binding to terephthalic acid to facilitate the separation from other by-products. However, since the use of the organic solvents necessarily causes environmental problems and requires additional purification processes, recently, methods without using organic solvents have been required.

### PRIOR ARTS

### Patent Documents

Korean Patent Registration No. 10-2593219.

### SUMMARY

In order to solve the problems of the above-described prior art, an object of the present disclosure is to provide a method for producing high-purity terephthalic acid with high efficiency by increasing an added value of plastic waste without using an organic solvent. Another object of the present disclosure is to provide a method for producing regenerated terephthalic acid capable of improving the yield of regenerated terephthalic acid and being environmentally friendly and economic by greatly shortening a process time through alkaline catalysts and sonication. Yet another object of the present disclosure is to provide a solution more suitable for commercial-scale recycling processes compared to conventional methods for recovering regenerated terephthalic acid using organic solvents.

In order to achieve the above-described objects, according to one embodiment of the present disclosure, there is provided a method for producing regenerated terephthalic acid by not using an organic solvent and regenerated terephthalic acid produced thereby, including mixing waste polyester and an alkaline catalyst, heating the mixture to 80 to 100° C. and sonicating the mixture at a frequency of 20 to 60 kHz and a power of 100 to 5,000 W under 1 atm conditions for 30 minutes to 5 hours, separating a terephthalic acid metal salt by adding water to the sonicated mixture, and treating the separated terephthalic acid metal salt with an acidic solution to precipitate regenerated terephthalic acid.

According to the present disclosure, the method for producing regenerated terephthalic acid can separate terephthalic acid from waste polyester with high efficiency in a short period of time by recovering terephthalic acid from waste polyester using alkaline catalysts and ultrasonic irradiation without using organic solvents. In addition, according to the present disclosure, a separate impurity removal process is not required by not using organic solvents such as toluene or xylene, and thus it is possible to shorten the reaction time and be economical, and there is an effect of increasing the production efficiency of regenerated terephthalic acid by accelerating the reactions and promoting uniform decomposition through ultrasonic irradiation.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF THE DRAWING

The above and other aspects, features and other advantages of the present disclosure will be more clearly understood from the following detailed description taken in conjunction with the accompanying drawings, in which:

FIG. 1 is a schematic view of a production method according to an example of the present disclosure.

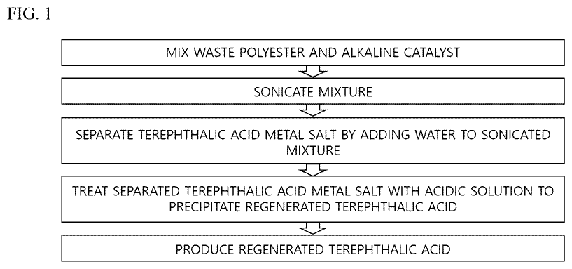

### DETAILED DESCRIPTION

Hereinafter, various examples of the present disclosure will be described in detail with reference to the accompanying drawings. The present disclosure is not limited to specific embodiments, and it should be understood to include various modifications, equivalents, and/or alternatives to the examples of the present disclosure. In connection with the description of the drawings, similar reference numerals may be used for similar components.

In this specification, expressions such as “have”, “may have”, “include”, or “may include” refer to the presence of the corresponding feature (e.g., components such as numerical values, functions, operations, or components), and do not exclude the presence of additional features.

In the present disclosure, the expression such as “A or B”, “at least one of A or/and B”, or “one or more of A or/and B” may include all possible combinations of items listed together. For example, “A or B”, “at least one of A or/and B”, or “one or more of A or/and B” may refer to all cases of (1) including at least one A, (2) including at least one B, or (3) including both at least one A and at least one B.

The expression of “configured to” used in the present disclosure may be changed and used to, for example, “suitable for”, “having the capacity to”, “designed to”, “adapted to”, “made to” or “capable of”, depending on a situation. The term of “configured to” does not necessarily mean only “specially designed to”.

The terms used in the present disclosure are used to illustrate only specific examples and may not be intended to limit the scope of other examples. A singular expression may include a plural expression unless the context clearly indicates otherwise. The terms used herein, including technical or scientific terms, may have the same meaning as generally understood by those skilled in the art described in the present disclosure. The terms defined in a general dictionary among the terms used in the present disclosure may be interpreted in the same or similar meaning as or to the meaning on the context of the related art, and will not be interpreted as an ideal or excessively formal meaning unless otherwise defined in the present disclosure. In some cases, even the terms defined in the present disclosure may not be interpreted to exclude the examples of the present disclosure.

The examples disclosed in the present disclosure are presented for explanation and understanding of the disclosed technical contents, and do not limit the scope of the present disclosure. Therefore, the scope of the present disclosure should be interpreted as including all changes or various other embodiments based on the technical idea of the present disclosure.

Hereinafter, preferred examples of the present disclosure will be described in detail. Terms and words used in the present specification and claims should not be interpreted as being limited to typical or dictionary meanings, but should be interpreted as having meanings and concepts which comply with the technical spirit of the present disclosure, based on the principle that the present inventors may appropriately define the concept of the term to describe their own invention in the best manner.

Therefore, the configurations of the examples described in the present specification are merely the most preferred embodiment of the present disclosure and are not intended to represent all of the technical ideas of the present disclosure, and thus, it should be understood that there are various equivalents and modifications capable of replacing the configurations at the time of this application.

Throughout the specification, when a part “includes” a component, unless otherwise specifically stated, it is meant to further include other components rather than excluding other components.

Hereinafter, the present disclosure will be described in detail.

A method for producing regenerated terephthalic acid characterized by not using an organic solvent according to an example of the present disclosure may include mixing waste polyester and an alkaline catalyst, heating the mixture to 80 to 100° C. and sonicating the mixture at a frequency of 20 to 60 kHz and a power of 100 to 5,000 W under 1 atm conditions for 30 minutes to 5 hours, separating a terephthalic acid metal salt by adding water to the sonicated mixture, and treating the separated terephthalic acid metal salt with an acidic solution to precipitate regenerated terephthalic acid. Hereinafter, each step will be described in detail. The step of mixing the waste polyester and the alkaline catalyst is to expand the structure of the waste polyester using the alkaline catalyst and to increase a bubble permeability effect that allows microbubbles generated in the subsequent sonication step to more easily penetrate into the polymer, thereby increasing the recovery rate of terephthalic acid.

The polyester is a polymer having an ester (RO—C(=O)—R′) chemical functional group in a main chain, which is also called polyester.

The polyester may be a polymer formed by condensation polymerization of dicarboxylic acid and dialcohol. The dicarboxylic acid may be any one selected from the group consisting of terephthalic acid, naphthalene dicarboxylic acid, diphenyl dicarboxylic acid, diphenyl ether dicarboxylic acid, diphenyl sulfone dicarboxylic acid, diphenoxyethane dicarboxylic acid, succinic acid, adipic acid, sebacic acid, azelaic acid, decanedicarboxylic acid, cyclohexanedicarboxylic acid, trimellitic acid, and pyromellitic acid. Preferably, the dicarboxylic acid may be terephthalic acid. The dialcohol may be any one selected from the group consisting of ethylene glycol, trimethylene glycol, 1,2-propanediol, tetramethylene glycol, neopentyl glycol, hexamethylene glycol, decanmethylene glycol, dodecamethylene glycol, 1,4-cyclohexanedimethanol, diethylene glycol, triethylene glycol, tetraethylene glycol, polyethylene glycol, dipropylene glycol, tripropylene glycol, tetrapropylene glycol, polypropylene glycol, di(tetramethylene) glycol, tri (tetramethylene) glycol, polytetramethylene glycol, pentaerythritol, and 2,2-bis(4-β-hydroxyethoxyphenyl) propane. Preferably, the dialcohol may be ethylene glycol.

The polyester may be at least one selected from the group consisting of polyethylene terephthalate (PET), polypropylene terephthalate (PPT), polyglycolide or polyglycolic acid (PGA), polylactic acid (PLA), polycaprolactone (PCL), polyhydroxyalkanoate (PHA), polyhydroxybutyrate (PHB), polyethylene adipate (PEA), polybutylene succinate (PBS), poly(3-hydroxybutyrate-co-3-hydroxyvalerate) (PHBV), polybutylene terephthalate (PBT), polytrimethylene terephthalate (PTT), polyethylene naphthalate (PEN), and Vectran. Preferably, the polyester may be polyethylene terephthalate (PET).

In the step of mixing the waste polyester and the alkaline catalyst, the alkaline catalyst may serve to penetrate between polyester chains to hydrolyze ester bonds and simultaneously weaken hydrogen bonds between the polymer chains.

In the step of mixing the waste polyester and the alkaline catalyst, the alkaline catalyst may be at least one selected from the group consisting of LiOH, NaOH, KOH, RbOH, CsOH, Ca(OH)₂, Sr(OH)₂, and Ba(OH)₂. Preferably, the alkaline catalyst may be at least one selected from the group consisting of NaOH and KOH. When the KOH is used as the alkaline catalyst, KOH may act as a strong Lewis base to promote the hydrolysis of ester bonds, and K⁺ ions are characterized by forming a more effective and stable chelate complex with terephthalic acid compared to Na⁺ ions to have an excellent chelation effect.

In the step of mixing the waste polyester and the alkaline catalyst, the weight ratio of the waste polyester and the alkaline catalyst may be 1:1 to 10. Preferably, the weight ratio may be 1:3 to 7. When the alkaline catalyst is below the numerical range, the hydrolysis of the ester bonds of the waste polyester is insufficient, making it difficult to structurally expand the polyester. Even if ultrasonic waves are irradiated to structurally unexpanded waste polyester, microbubbles caused by ultrasonic waves may be difficult to penetrate between polyester structures, and the terephthalic acid recovery rate may be reduced. When the alkaline catalyst is below the numerical range, a process of separating the alkaline catalyst may be added, which is not economical and may lower the recovery efficiency of terephthalic acid.

The step of heating the mixture to 80 to 100° C. and sonicating the mixture at a frequency of 20 to 60 kHz and a power of 100 to 5,000 W under 1 atm conditions for 30 minutes to 5 hours is to further sonicating waste polyester structurally weakened by the alkaline catalyst to separate terephthalic acid with high purity.

The sonication has effects of cutting the molecular chains of waste polyester and increasing the surface area, thereby promoting the hydrolysis of the alkaline catalyst and increasing the recovery efficiency of terephthalic acid. The ultrasonic waves cause a cavitation phenomenon in a liquid, causing repeated expansion and condensation of microbubbles, and when the bubbles collapse, a local environment of high temperature and high pressure is formed. As a result, there is an effect of rapidly breaking down the ester bonds of polyester and significantly accelerating the reaction rate. In particular, the local environment of high temperature and high pressure formed by the cavitation effect enables depolymerization of polyester at a lower temperature and in a shorter time than conventional methods for recovering regenerated terephthalic acid by replacing high energy required in the alkaline hydrolysis process. It is possible to implement an economical and efficient process for recovering regenerated terephthalic acid by reducing energy consumption and shortening process time through sonication.

In the step of heating the mixture to 80 to 100° C. and sonicating the mixture at a frequency of 20 to 60 kHz and a power of 100 to 5,000 W under 1 atm conditions for 30 minutes to 5 hours, when sonicating the mixture at a frequency below the numerical range or sonicating the mixture at a power below the numerical range, it is difficult for microbubbles caused by ultrasonic waves to penetrate between the molecular chains of waste polyester and it is not easy to create a local environment of high temperature and high pressure, so that the regenerated terephthalic acid may contain a large amount of impurities and the reaction time may be increased. When sonicating at a frequency above the numerical range or sonicating at a power above the numerical range, the expansion and condensation of bubbles are repeated excessively to increase the reaction temperature and reduce economic efficiency due to excessive energy consumption. When the ultrasonic waves are irradiated for the time below numerical range, the waste polyester is not structurally sufficiently expanded, making it difficult to obtain terephthalic acid with high purity, and when the ultrasonic waves are irradiated for the time above numerical range, there is a problem that the economic efficiency is reduced due to an energy-consuming process. However, depending on an amount of reaction solvent or reaction solute, the ultrasonic power and treatment time may increase linearly or nonlinearly.

The step of separating the terephthalic acid metal salt by adding water to the sonicated mixture may promote the monomerization and hydrolysis of the polyester, and efficiently remove water-soluble impurities because water is added, and is economical without additionally performing no subsequent purification process. Furthermore, the crystallization of the terephthalic acid metal salt may be promoted to increase the recovery efficiency of pure regenerated terephthalic acid. The metal salt varies depending on a type of alkaline catalyst, and the terephthalic acid metal salt may include at least one selected from the group consisting of lithium terephthalic acid salt (Li₂-TPA), sodium terephthalic acid salt (Na₂-TPA), potassium terephthalic acid salt (K₂-TPA), rubidium terephthalic acid salt (Rb₂-TPA), cesium terephthalic acid salt (Cs₂-TPA), calcium terephthalic acid salt (Ca-TPA), strontium terephthalic acid salt (Sr-TPA), and barium terephthalic acid salt (Ba-TPA).

In the step of separating the terephthalic acid metal salt by adding water to the sonicated mixture, the weight ratio of the sonicated mixture and water may be 1:0.1 to 10. When the water content is below the numerical range, it may be difficult to separate the terephthalic acid metal salt, and when the water content is above the numerical range, the concentration of the terephthalic acid metal salt may be too low to reduce the separation efficiency.

In the step of separating the terephthalic acid metal salt by adding water to the sonicated mixture, the terephthalic acid metal salt may be included in an amount of 10 to 50 wt % based on the total weight of the solution in which the sonicated mixture and water are mixed. When the content of the terephthalic acid metal salt is below the numerical range, the concentration of the metal salt may be too low, which may reduce the separation efficiency, and the solution is diluted, which may increase energy consumption in the subsequent process. When the content of the terephthalic acid metal salt is above the numerical range, the solubility limit of the terephthalic acid metal salt is reached, so that precipitation occurs, and the viscosity of the solution increases, thereby making it difficult to be separated and lowering the efficiency of removing impurities.

In the step of separating the terephthalic acid metal salt by adding water to the sonicated mixture, the water may be added in an amount of 5 to 15 times based on the weight of the waste polyester in the step of mixing the waste polyester and the alkaline catalyst. When the water is added below the numerical range, effective dispersion of the alkaline catalyst may not be achieved and the solubility of the terephthalic acid metal salt may not be sufficiently secured, which may lower the separation efficiency. When the water is added above the numerical range, the reaction mixture becomes diluted, so that the reaction rate slows down, and the efficiency of the process decreases, and the efficiency of the subsequent separation process decreases, thereby making it difficult to recover high-purity regenerated terephthalic acid.

The step of treating the separated terephthalic acid metal salt with the acidic solution to precipitate the regenerated terephthalic acid is to precipitate pure regenerated terephthalic acid by separating metal ions from the terephthalic acid metal salt through treatment with the acidic solution.

In the step of treating the separated terephthalic acid metal salt with the acidic solution to precipitate the regenerated terephthalic acid, the weight ratio of the separated terephthalic acid metal salt and the acidic solution may be 1:0.5 to 2. Since the acidic solution causes a change in pH to promote the crystallization of terephthalic acid and enable the recovery of pure terephthalic acid, when the acidic solution is treated below the numerical range, complete decomposition of the terephthalic acid metal salt may not occur and the purity may decrease, and when the acidic solution is treated above the numerical range, side reactions may occur to generate impurities, and the structure of terephthalic acid may be modified due to excessive acidic conditions.

In the step of treating the separated terephthalic acid metal salt with the acidic solution to precipitate the regenerated terephthalic acid, the acidic solution may be at least one selected from the group consisting of hydrochloric acid, sulfuric acid, nitric acid, phosphoric acid, acetic acid, oxalic acid, citric acid, tartaric acid, maleic acid, and succinic acid. Preferably, the acidic solution may be at least one selected from the group consisting of hydrochloric acid, sulfuric acid and nitric acid.

The purity of the regenerated terephthalic acid may be 98% or higher.

According to another example of the present disclosure, there is provided regenerated terephthalic acid produced by any one of the production methods.

Hereinafter, the present disclosure will be described in more detail through examples.

These Examples are only to describe the present disclosure in more detail, and it will be apparent to those skilled in the art that the scope of the present disclosure is not limited by these Examples in accordance with the gist of the present disclosure.

### EXAMPLES AND COMPARATIVE EXAMPLES

### Example 1

420 g of PET waste, which was washed to remove surface impurities and crushed, was mixed with 2,723 g of a 45% NaOH aqueous solution (S1).

A beaker containing the mixture was placed in a reactor attached with an ultrasonic generator at a frequency of 28 kHz and a power of 500 W, heated to 95° C. under 1 atm conditions, and then sonicated for 90 minutes while stirring at 200 rpm using a mechanical overhead stirrer (S2).

628.6 g of a sodium terephthalic acid salt (Na₂-TPA) was separated by adding 5,040 g of water to the sonicated mixture (S3).

The separated sodium terephthalic acid salt (Na₂-TPA) was treated with 450 g of sulfuric acid to precipitate terephthalic acid (S4).

### Examples 2 to 6

Terephthalic acid was precipitated in the same manner as Example 1, except that process conditions for each step in [Table 1] below were changed.

TABLE 1

| | | Alkaline | Terephthalic |
|---|---|---|---|
| | Alkaline catalyst type | catalyst content | acid metal |
| | in step S1 | (g) in step S1 | salt in step S3 |
| Example 1 | 45% NaOH aqueous | 2,723 | Na2-TPA |
| | solution | | |
| Example 2 | 45% NaOH aqueous | 420 | Na₂-TPA |
| | solution | | |
| Example 3 | 45% NaOH aqueous | 1,260 | Na₂-TPA |
| | solution | | |
| Example 4 | 45% NaOH aqueous | 2,940 | Na₂-TPA |
| | solution | | |
| Example 5 | 45% NaOH aqueous | 4,200 | Na₂-TPA |
| | solution | | |
| Example 6 | 45% KOH aqueous solution | 2,723 | K₂-TPA |

### Examples 7 to 22

Terephthalic acid was precipitated in the same manner as Example 1, except that process conditions for each step in [Table 2] below were changed.

TABLE 2

| | Frequency | Sonication | Added | Acidic |
|---|---|---|---|---|
| | (kHz) in | time (min) | water (g) | solution (g) |
| | step S2 | in step S2 | in step S3 | in step S4 |
| Example 7 | 15 | 90 | 5,040 | 450 |
| Example 8 | 20 | 90 | 5,040 | 450 |
| Example 9 | 60 | 90 | 5,040 | 450 |
| Example 10 | 75 | 90 | 5,040 | 450 |
| Example 11 | 28 | 10 | 5,040 | 450 |
| Example 12 | 28 | 30 | 5,040 | 450 |
| Example 13 | 28 | 120 | 5,040 | 450 |
| Example 14 | 28 | 150 | 5,040 | 450 |
| Example 15 | 28 | 90 | 1,680 | 450 |
| Example 16 | 28 | 90 | 2,100 | 450 |
| Example 17 | 28 | 90 | 6,300 | 450 |
| Example 18 | 28 | 90 | 7,140 | 450 |
| Example 19 | 28 | 90 | 5,040 | 250 |
| Example 20 | 28 | 90 | 5,040 | 310 |
| Example 21 | 28 | 90 | 5,040 | 1,250 |
| Example 22 | 28 | 90 | 5,040 | 1,570 |

### Comparative Example 1

Comparative Example 1 was performed in the same manner as Example 1, except that 2,723 g of a 45% NaOH aqueous solution was not treated in step S1 of Example 1.

### Comparative Example 2

Comparative Example 2 was performed in the same manner as Example 1, except that step S2 of Example 1 above was not performed.

### Comparative Example 3

Comparative Example 3 was performed in the same manner as Example 1, except that toluene as an organic solvent was treated instead of 2,723 g of the 45% NaOH aqueous solution in step S1 of Example 1.

### Experimental Example 1

The conversion rate (%) was measured for regenerated terephthalic acid according to Examples 1 to 22 and Comparative Examples 1 and 2. The conversion rate (%) of regenerated terephthalic acid was calculated with a weight ratio of recovered terephthalic acid to added PET waste.

TABLE 3

| | Unreacted | Convention |
|---|---|---|
| | PET pieces (g) | rate (%) |
| Example 1 | 1.2 | 99.7 |
| Example 2 | 133.3 | 68.3 |
| Example 3 | 74.5 | 82.3 |
| Example 4 | 21.9 | 94.8 |
| Example 5 | 27.4 | 93.5 |
| Example 6 | 13.8 | 96.7 |
| Example 7 | 7.3 | 98.3 |
| Example 8 | 9.1 | 97.8 |
| Example 9 | 118.2 | 71.9 |
| Example 10 | 148.9 | 64.5 |
| Example 11 | 280.8 | 33.1 |
| Example 12 | 137.2 | 67.3 |
| Example 13 | 1.8 | 99.8 |
| Example 14 | 1.6 | 99.6 |
| Example 15 | 1.8 | 99.6 |
| Example 16 | 1.7 | 99.6 |
| Example 17 | 2.1 | 99.5 |
| Example 18 | 1.7 | 99.6 |
| Example 19 | 2 | 99.5 |
| Example 20 | 1.7 | 99.6 |
| Example 21 | 1.9 | 99.5 |
| Example 22 | 2.3 | 99.5 |
| Comparative Example 1 | 419.1 | 0.2 |
| Comparative Example 2 | 401.2 | 4.5 |
| Comparative Example 3 | 419.7 | 0.1 |

### Experimental Example 2

The yield of regenerated terephthalic acid according to Examples 1 to 22 and Comparative Examples 1 and 2 was calculated with the weight ratio of the recovered terephthalic acid to the added PET waste, and the purity was measured using high-performance liquid chromatography (HPLC). The results were shown in [Table 4] below.

TABLE 4

| | Yield (%) | Purity (%) |
|---|---|---|
| Example 1 | 92.3 | 99.6 |
| Example 2 | 59.7 | 97.5 |
| Example 3 | 74.2 | 98.9 |
| Example 4 | 86.4 | 98.2 |
| Example 5 | 87.8 | 97.8 |
| Example 6 | 89.4 | 98.2 |
| Example 7 | 87.2 | 94.7 |
| Example 8 | 88.3 | 96.2 |
| Example 9 | 60.1 | 97.2 |
| Example 10 | 56.5 | 94.8 |
| Example 11 | 18.2 | 92.3 |
| Example 12 | 53.5 | 94.8 |
| Example 13 | 91.7 | 95.6 |
| Example 14 | 90.3 | 94.4 |
| Example 15 | 85.6 | 98.7 |
| Example 16 | 89.3 | 99.0 |
| Example 17 | 92.1 | 99.4 |
| Example 18 | 92.2 | 99.3 |
| Example 19 | 42.1 | 97.8 |
| Example 20 | 60.7 | 98.3 |
| Example 21 | 92.1 | 98.3 |
| Example 22 | 92.0 | 98.0 |
| Comparative Example 1 | 0 | 0 |
| Comparative Example 2 | 2.1 | 94.3 |
| Comparative Example 3 | 0 | 0 |

1. A method for producing regenerated terephthalic acid characterized by not using an organic solvent, the method comprising:
mixing waste polyester and an alkaline catalyst;
heating the mixture to 80 to 100□C and sonicating the mixture at a frequency of 20 to 60 kHz and a power of 100 to 5,000 W under 1 atm conditions for 30 minutes to 5 hours;
separating a terephthalic acid metal salt by adding water to the sonicated mixture; and
treating the separated terephthalic acid metal salt with an acidic solution to precipitate regenerated terephthalic acid.

2. The method of claim 1, wherein in the step of mixing the waste polyester and the alkaline catalyst, the alkaline catalyst is at least one selected from the group consisting of LiOH, NaOH, KOH, RbOH, CsOH, Ca(OH)2, Sr(OH)2, and Ba(OH)2.

3. The method of claim 1, wherein in the step of mixing the waste polyester and the alkaline catalyst, the weight ratio of the waste polyester and the alkaline catalyst is 1:1 to 10.

4. The method of claim 1, wherein in the step of separating the terephthalic acid metal salt by adding water to the sonicated mixture, the weight ratio of the sonicated mixture and the water may be 1:0.1 to 10.

5. The method of claim 1, wherein in the step of separating the terephthalic acid metal salt by adding water to the sonicated mixture, the terephthalic acid metal salt is included in an amount of 10 to 50 wt % based on the total weight of the solution in which the sonicated mixture and the water are mixed.

6. The method of claim 1, wherein in the step of separating the terephthalic acid metal salt by adding water to the sonicated mixture, the water is added in an amount of 5 to 15 times based on the weight of the waste polyester in the step of mixing the waste polyester and the alkaline catalyst.

7. The method of claim 1, wherein in the step of treating the separated terephthalic acid metal salt with the acidic solution to precipitate the regenerated terephthalic acid, the weight ratio of the separated terephthalic acid metal salt and the acidic solution is 1:0.5 to 2.

8. The method of claim 1, wherein in the step of treating the separated terephthalic acid metal salt with the acidic solution to precipitate the regenerated terephthalic acid, the acidic solution is at least one selected from the group consisting of hydrochloric acid, sulfuric acid, nitric acid, phosphoric acid, acetic acid, oxalic acid, citric acid, tartaric acid, maleic acid, and succinic acid.

9. The method of claim 1, wherein the purity of the regenerated terephthalic acid is 98% or higher.

10. Regenerated terephthalic acid produced by a method characterized by not using an organic solvent, the method comprising:
mixing waste polyester and an alkaline catalyst;
heating the mixture to 80 to 100□C and sonicating the mixture at a frequency of 20 to 60 KHz and a power of 100 to 5,000 W under 1 atm conditions for 30 minutes to 5 hours;
separating a terephthalic acid metal salt by adding water to the sonicated mixture; and
treating the separated terephthalic acid metal salt with an acidic solution to precipitate regenerated terephthalic acid.

11. The regenerated terephthalic acid of claim 10, wherein in the step of mixing the waste polyester and the alkaline catalyst, the alkaline catalyst is at least one selected from the group consisting of LiOH, NaOH, KOH, RbOH, CsOH, Ca(OH)2, Sr(OH)2, and Ba(OH)2.

12. The regenerated terephthalic acid of claim 10, wherein in the step of mixing the waste polyester and the alkaline catalyst, the weight ratio of the waste polyester and the alkaline catalyst is 1:1 to 10.

13. The regenerated terephthalic acid of claim 10, wherein in the step of separating the terephthalic acid metal salt by adding water to the sonicated mixture, the weight ratio of the sonicated mixture and the water may be 1:0.1 to 10.

14. The regenerated terephthalic acid of claim 10, wherein in the step of separating the terephthalic acid metal salt by adding water to the sonicated mixture, the terephthalic acid metal salt is included in an amount of 10 to 50 wt % based on the total weight of the solution in which the sonicated mixture and the water are mixed.

15. The regenerated terephthalic acid of claim 10, wherein in the step of separating the terephthalic acid metal salt by adding water to the sonicated mixture, the water is added in an amount of 5 to 15 times based on the weight of the waste polyester in the step of mixing the waste polyester and the alkaline catalyst.

16. The regenerated terephthalic acid of claim 10, wherein in the step of treating the separated terephthalic acid metal salt with the acidic solution to precipitate the regenerated terephthalic acid, the weight ratio of the separated terephthalic acid metal salt and the acidic solution is 1:0.5 to 2.

17. The regenerated terephthalic acid of claim 10, wherein in the step of treating the separated terephthalic acid metal salt with the acidic solution to precipitate the regenerated terephthalic acid, the acidic solution is at least one selected from the group consisting of hydrochloric acid, sulfuric acid, nitric acid, phosphoric acid, acetic acid, oxalic acid, citric acid, tartaric acid, maleic acid, and succinic acid.

18. The regenerated terephthalic acid of claim 10, wherein the purity of the regenerated terephthalic acid is 98% or higher.
